(19) United States
(12) Patent Application Publication
(10) Pub. No.: US 2026/0207395 A1
(43) Pub. Date: Jul. 23, 2026

(54) AN ABSORBENT ARTICLE HAVING STANDING GATHERS AND LEG CUFFS

(71) Applicant: Essity Hygiene and Health Aktiebolag, Göteborg (SE)

(72) Inventors: Helena CORNELIUSSON, Göteborg (SE); Lisa NILSSON, Göteborg (SE); Emelie ADIELS, Göteborg (SE); Yasha ASAYESH, Göteborg (SE)

(73) Assignee: Essity Hygiene and Health Aktiebolag, Göteborg (SE)

(21) Appl. No.: 19/140,621

(22) PCT Filed: Dec. 20, 2022
(86) PCT No.: PCT/EP2022/087093

§ 371 Date: Jun. 18, 2025

(51) Int. Cl. A61F 13/494; A61F 13/49; A61F 13/514

(52) U.S. Cl. CPC A61F 13/49413; A61F 13/49014; A61F 13/49017; A61F 13/51478; A61F 2013/49092; A61F 2013/51452

(57) ABSTRACT

An absorbent article includes a liquid permeable topsheet, a liquid impermeable backsheet and an absorbent core. A nonwoven outer cover layer overlies the backsheet. A pair of nonwoven barrier cuff strips are attached to the topsheet along a standing gather hinge line dividing the strips in standing gather and leg cuff portions. The standing gather portions form part of a pair of elasticated standing gathers. The leg cuff portions form part of a pair of leg cuffs arranged outward of the standing gathers. A first part of the leg cuff portion is folded back on a second part along a leg cuff fold line, forming a leg cuff fold. Each fold contains pre-tensioned elastic elements extending inside and attached between the first and second part. A side portion of the outer cover layer overlaps with the leg cuff portion and is joined to the leg cuff fold.

### TECHNICAL FIELD

The disclosure pertains to an absorbent article having first and second waist edges and first and second side edges and comprising a liquid permeable topsheet, a liquid impermeable backsheet and an absorbent core disposed between the topsheet and the backsheet. The absorbent article is arranged to be worn in a panty-like manner with the waist edges forming a waist opening and the side edges forming leg openings.

A nonwoven outer cover layer overlays the backsheet and forms an outer surface of the absorbent article. The article further comprises a pair of elasticated standing gathers and a pair of elasticated leg cuffs.

### BACKGROUND

Disposable absorbent articles, such as disposable diapers which are applied to the lower body of a wearer and fastened around the wearer's body as a pair of underpants are commonly provided with elastic elements to create fluid barriers and to improve body conformance of the articles. A good body-conforming fit of a disposable absorbent article around the waist and legs of a wearer of the article ensures that the article is held in close contact with the body of the wearer, thereby reducing the risk of fluid leaking out of the absorbent article. Additional leakage protection may be obtained by arranging elasticated standing gathers, also known as “raised barriers” or “inner barriers” inside the absorbent article.

A further important aspect of articles of this kind is that they are soft and comfortable to wear and that they do not give rise to chafing of the sensitive skin in the genital area. A further concern is that tensioned elastic elements may cause discomfort and red-marking by exerting a high pressure on the skin of a wearer, in particular around the legs of the wearer.

### SUMMARY

Disposable absorbent articles having good leakage security and comfort may be achieved by the features of claim 1. Variations of the disclosure are set out in the dependent claims.

Disclosed herein is an absorbent article having a first waist edge and a second waist edge extending in a transverse direction of the absorbent article and a first side edge and a second side edge extending in a longitudinal direction of the absorbent article, the absorbent article comprising:
a liquid permeable topsheet;
a liquid impermeable backsheet;
an absorbent core disposed between the topsheet and the backsheet;
a nonwoven outer cover layer overlaying the backsheet and forming an outer surface of the absorbent article;
a pair of nonwoven barrier cuff strips extending in the longitudinal direction and being symmetrically arranged with one barrier cuff strip on each side of a longitudinal centre line, each nonwoven barrier cuff strip comprising a standing gather portion and a leg cuff portion, the standing gather portion being defined between a free standing gather edge extending in the longitudinal direction and a standing gather hinge line extending in the longitudinal direction, the barrier cuff strip being attached to the topsheet along the standing gather hinge line and having one or more elastic elements arranged along the free standing gather edge;
a pair of standing gathers comprising the standing gather portions of the barrier cuff strips;
a pair of leg cuffs extending in the longitudinal direction and being symmetrically arranged along the first and second side edges, each leg cuff being arranged outward of the standing gather portion of the corresponding barrier cuff strip in the transverse direction; and wherein each leg cuff comprises:
the leg cuff portion of the corresponding nonwoven barrier cuff strip, the leg cuff portion of the nonwoven barrier cuff strip extending in the transverse direction between the standing gather hinge line and a leg cuff fold line along which the nonwoven barrier cuff strip is folded back on itself and forms a leg cuff fold, the leg cuff fold line extending in the longitudinal direction and dividing the leg cuff portion of the barrier cuff strip in a first part and a second part which first and second parts together form the leg cuff fold;
a plurality of spaced-apart pre-tensioned elastic elements extending in the longitudinal direction inside the leg cuff fold and being attached between the first part and the second part of the leg cuff fold; and
a side portion of the outer cover layer overlapping with the leg cuff portion of the nonwoven barrier cuff strip and being joined to the leg cuff fold with a side edge of the side portion of the outer cover layer being arranged at a distance from the leg cuff fold line.

An article as disclosed herein may be in the form of an open diaper comprising a liquid permeable topsheet, a liquid impermeable backsheet and an absorbent core disposed between the topsheet and the backsheet.

An open diaper as disclosed herein may comprise a pair of side flaps arranged at one of the first or second waist edges, each side flap extending in the transverse direction from a proximal attached end to a free distal end and comprising a fastener being arranged at the distal end of the side flap.

An open diaper may alternatively be a belted diaper which is closed by connecting the ends of a belt provided at a waist edge of the article with each other.

An absorbent article as disclosed herein may be a disposable article which is intended to be discarded after a single use. The absorbent article may be an absorbent insert which is arranged for use together with a reusable holder. Typically, the reusable holder incorporates fastening means for fastening the combination of the absorbent article and the holder in the manner of a pair of panties around the trunk of a wearer of the article.

The absorbent articles as disclosed herein are arranged to be worn about the lower torso of a wearer and comprises a liquid permeable topsheet, a liquid impermeable backsheet and an absorbent core sandwiched therebetween. A nonwoven outer cover layer overlies the backsheet on an outer surface of the absorbent article. A pair of nonwoven barrier cuff strips are attached to the topsheet along a standing gather hinge line which divides the barrier cuff strips in a standing gather portion and a leg cuff portion. The standing gather portions of the barrier cuff strips form part of a pair of elasticated standing gathers. The leg cuff portions of the barrier cuff strips form part of a pair of leg cuffs which are arranged outward of the standing gathers. A first part of the leg cuff portion of each nonwoven barrier cuff strip is folded back on a second part of the leg cuff portion of the barrier cuff strip along a leg cuff fold line so that a leg cuff fold is formed between the first and second parts of the leg cuff portion of the barrier cuff strip. Each leg cuff fold contains a plurality of spaced-apart pre-tensioned elastic elements extending inside the leg cuff fold and being attached between the first part and the second part of the leg cuff fold. A side portion of the outer cover layer overlaps with the leg cuff portion of the nonwoven barrier cuff strip and is directly or indirectly joined to the leg cuff fold with a side edge of the side portion of the outer cover layer being arranged at a distance from the leg cuff fold line. By arranging the outer cover layer at a distance from the leg cuff fold line the leg cuff may pe perceived as softer since there is no free material edge at the outermost position i.e. the outer leg cuff side edge.

The distance between the side portion of the outer cover layer and the leg cuff fold line may be at least 2 mm, preferably at least 5 mm, most preferably at least 10 mm.

The absorbent article has a nonwoven outer cover layer which overlies the liquid impermeable backsheet and forms an outer textile-like surface of the absorbent article. The side portions of the nonwoven outer cover layer may extend in the transverse direction laterally outside the liquid impermeable backsheet. Alternatively, the nonwoven outer cover and the backsheet may have the same width. In the latter case the nonwoven outer cover and the backsheet may be prelaminated whereas when the backsheet is narrower than the nonwoven outer cover it may be laminated in line during manufacturing of the absorbent article.

If the liquid impermeable backsheet and the outer cover layer has the same width, they may be arranged with a respective side edge being arranged at the same distance from the leg cuff fold line which is the outer leg cuff side edge. The distance may be at least 2 mm, preferably at least 5 mm, most preferably at least 10 mm.

A pair of nonwoven barrier cuff strips are arranged with a standing gather portion of each barrier cuff strip forming part of a standing gather and a leg cuff portion of each barrier cuff strip forming part of an elastic leg cuff. The leg cuffs are arranged laterally outward of the standing gathers in the transverse direction. Each barrier cuff strip is attached to the topsheet along a standing gather hinge line which also divides the barrier cuff strip in the standing gather portion and the leg cuff portion.

The barrier cuff strips are directly attached to the topsheet along the standing gather hinge lines, without any intervening components between the barrier cuff strips and the topsheet.

An article as disclosed herein is skin-friendly and comfortable to wear as all body-contacting surfaces of the article are constituted by soft nonwoven materials. The leg cuffs are particularly soft as the pretensioned elastic elements in the leg cuffs are arranged inside the leg cuff folds, providing the leg cuffs with a folded outer edge. The plurality of spaced-apart pre-tensioned elastic elements cause the nonwoven material in the leg cuff folds to contract and form soft, airy, and evenly distributed wrinkles on the surface of the leg cuff. The wrinkled elasticated portion of the leg cuff acts as a cushioning distance means between a wearer's body and the elastic elements. Thus, under the influence of the contractive forces from the elastic elements which are distributed over the width of each leg cuff, the leg cuff portion of the nonwoven barrier cuff strip form a soft, bulky, yet highly body conforming leg cuff.

When the article is worn, an upper part of the leg cuff fold will be a wearer-facing part of the leg cuff fold and a lower part of the leg cuff fold will be a garment-facing part of the leg cuff fold.

The first part of each barrier cuff strip may be folded back along the leg cuff fold line on the wearer-facing side of the absorbent article. In this fold configuration, the first part of the barrier cuff strip is folded on top of the second part of the barrier cuff strip and is attached to the second part of the barrier cuff strip on the wearer-facing side of the absorbent article.

Alternatively, the first part of each barrier cuff strip may be folded back along the leg cuff fold line on the garment-facing side of the absorbent article. In this fold configuration, the first part of the barrier cuff strip is folded below the second part of the barrier cuff strip and is attached to the second part of the barrier cuff strip on the garment-facing side of the absorbent article. In such a configuration an inner portion of the first part of the leg cuff portion of each barrier cuff strip may overlap the liquid permeable topsheet such that the leg cuff edge of the leg cuff portion of the barrier cuff strip is arranged inwards in relation to the side edge of the topsheet in transverse direction. The leg cuff edge of the leg cuff portion of the barrier cuff strip may be arranged between the second part of the leg cuff and the topsheet or between the topsheet and the backsheet.

The side edge of the side portion of the outer cover layer is arranged at a distance from the leg cuff fold line in the transverse direction. A leg edge portion of each leg cuff fold which is located between the side edge of the side portion of the outer cover layer and the leg cuff fold line may contain two or more of the elastic elements of the plurality of elastic elements in the leg cuff fold. The leg edge portion of each leg cuff fold which extends in the transverse direction outside the corresponding side edge of the side portion of the outer cover layer constitutes a portion of the leg cuff which is more susceptible to deformation than a portion of the leg cuff where the leg cuff fold is attached to the outer cover layer and/or to the liquid impermeable backsheet. Thereby, the nonwoven material in the leg edge portion of the leg cuff can be gathered in the longitudinal direction by a lower contractive force than required for attaining the same degree of gathering in the portion of the leg cuff where the leg cuff fold is attached to the outer cover layer and/or to the liquid impermeable backsheet. Furthermore, the leg edge portion of the leg cuff has a higher compressibility in the transverse direction than the portion of the leg cuff where the leg cuff fold is attached to the outer cover layer and/or to the liquid impermeable backsheet. Thereby, the leg edge portion of the leg cuff provides lateral compressibility of the leg cuff which contributes to a cushioning effect against a wearer's legs.

In an absorbent article as disclosed herein, the leg cuffs may be relatively wide. Each leg cuff may have a major part of the width of the leg cuff being elasticated by the plurality of elastic elements. A width of the leg cuff may be in the range of from 20 millimetres to 60 millimetres, preferably from 30 millimetres to 60 millimetres. The elastic elements of the plurality of elastic elements may be distributed over an area of the leg cuff having a width which is at least 40% of the width of the leg cuff, preferably at least 50% of the width of the leg cuff.

In an absorbent article as disclosed herein, the plurality of elastic elements may be constituted by at least 3 elastic elements, preferably at least 4 elastic elements. The plurality of elastic elements may be constituted by 3 to 15 elastic elements, preferably 4 to 10 elastic elements, more preferred 5 to 10 elastic elements and most preferred 5 to 7 elastic elements.

The elastic elements may be elastic threads, strings or elastic bands.

The elastic elements in the plurality of elastic elements in each leg cuff may be spaced apart in the transverse direction by from 1 millimetre to 8 millimetres, preferably from 3 millimetre to 8 millimetres. The elastic elements may be applied with equal spacing over the elasticated area of the leg cuff or may be applied with different spacing over the width of the leg cuff. By way of example, the elastic elements may be applied with a smaller spacing in an inner part of the leg cuff than in an outer part of the leg cuff to achieve a tighter fit along the inner parts of the leg cuffs.

In an absorbent article as disclosed herein, the elasticated part of each leg cuff may have a width of from 20 millimetres to 40 millimetres, preferably 20 millimetres to 35 millimetres as measured in the transverse direction between the outermost elastic element of the plurality of elastic elements and the innermost elastic element of the plurality of elastic elements.

In a leg cuff in an absorbent article as disclosed herein, the elasticated part of each leg cuff may have a width of from 20 millimetres to 60 millimetres as measured in the transverse direction between the outermost elastic element of the plurality of elastic elements and the innermost elastic element of the plurality of elastic elements, a number of elastic elements in the elasticated part of the leg cuff within the range of from 4 to 7 and a spacing between the elastic elements of from 4 to 6 millimetres.

The leg cuff folds may be arranged such that there is no overlap between the leg cuff folds and the topsheet. If the backsheet and the outer cover layer have the same width in the transverse direction, the leg cuff folds may then be directly attached to the liquid impermeable backsheet. If the outer cover layer has a greater extension in the transverse direction than the backsheet, the leg cuff folds may instead be directly attached to the outer cover layer. Accordingly, the outer cover layer may be directly joined to the leg cuff folds without any intervening component or may be indirectly joined to the leg cuff folds by being joined to the intervening backsheet. By not involving the topsheet in the direct or indirect join between the leg cuff folds and the outer cover layer, the number of material layers and attachments between layers in the inner part of each leg cuff can be reduced. Thereby, a reduction of the flexibility and conformability of the inner part of the leg cuff may be avoided.

Alternatively, the leg cuff folds may be arranged such that there is an overlap between the leg cuff folds and the topsheet. Such an arrangement may reduce the risk of the leg cuff folds detaching and exposing the elastic elements.

In an absorbent article as disclosed herein, all or substantially all elastic elements of the plurality of elastic elements in each leg cuff fold may be arranged in an outer part of the leg cuff fold which is free from overlap with the liquid impermeable backsheet. Thereby, the elasticated parts of the leg cuffs are highly breathable, flexible and contractible.

The nonwoven materials used for the outer cover material and the barrier cuff strip may include one or more spunbond layers and optionally one or more meltblown layers. The nonwoven material in the nonwoven barrier strip may be different from the nonwoven material of the outer cover material from which the leg cuff fold is formed. The nonwoven barrier cuff strip may be formed from a relatively bulky material. The basis weight of the material in the nonwoven barrier strip may be in the range of from 10 g/m² to 25 g/m² . The nonwoven material may be made of thermoplastic polymer fibers or filaments, such as polyolefin, polyesters, polyamide and combinations thereof. The nonwoven material may be formed by any of a variety of different processes, such as spunbonding, airlaying, meltblowing or bonded carded web formation processes. The nonwoven layer may be made of co-formed lamina of nonwoven materials such as an SMS (spunbond/meltblown/spunbond) nonwoven material or an SS (spunbond/spunbond) nonwoven material. The thermoplastic polymer materials in the nonwoven layer may be polypropylene or bicomponent fibers of polypropylene and polyethylene, or of a combination of such materials. It may be preferred that the nonwoven barrier strip is constituted by a hydrophobic material. The nonwoven barrier cuff strip may be a single layer nonwoven material.

The pre-tensioned elastic element or elastic elements which is/are arranged along the free standing gather edge of the nonwoven barrier cuff strip may be arranged in a folded-over portion of the nonwoven barrier cuff strip at the free standing gather edge. The pre-tensioned elastic element or elastic elements of the standing gather portion of the nonwoven barrier cuff strip may comprise any suitable number of pre-tensioned elastic elements. However, the pre-tensioned elastic element or elastic elements are preferably 1, 2 or 3 elastic elements arranged parallel to each other in an edge portion of the standing gather portion of the nonwoven barrier cuff strip. The spacing between the elastic elements on the standing gather portion of the nonwoven barrier cuff strip may be 2 to 5 millimetres. The elastic element or elements may be in the form of one or more elastic thread, string or band.

In an absorbent article as disclosed herein, the elastic elements of the plurality of elastic elements in the leg cuff portion of each barrier cuff strip may be attached between the first and second parts of the leg cuff fold by means of adhesive. The adhesive may be applied to the elastic elements, whereby the first and second parts of the leg cuff fold are indirectly attached to each other by means of the elastic elements. Alternatively, or in addition thereto, the adhesive may be applied to one or both of the first part and the second part of the leg cuff fold.

In an absorbent article as disclosed herein, one or more elastic elements being arranged in an inner part of each leg cuff fold may have a greater tensile resistance than one or more elastic elements being arranged in an outer part of the leg cuff fold. Alternatively, or in addition thereto, one or more elastic elements being arranged in an inner part of the leg cuff fold may be arranged with a smaller spacing than elastic elements being arranged in an outer part of the leg cuff fold.

The one or more elastic elements being arranged in the inner part of each leg cuff fold may have a linear density (dtex) which is higher than the linear density of the one or more elastic elements being arranged in the outer portion of the leg cuff fold.

The elastic elements of the plurality of elastic elements in each leg cuff are preferably applied with an outermost elastic element of the plurality of elastic elements being arranged not more than 5 millimetres from the fold-line in the leg cuff fold, preferably not more than 3 millimetres from the fold-line in the leg cuff fold and most preferred not more than 1 millimetre from the fold-line in the leg cuff fold. The outermost elastic element in each leg cuff may be arranged such that there is no distance between the fold-line in the leg cuff fold and the outermost elastic element.

The leg cuffs of the absorbent articles as disclosed herein are well-defined, neat and distinct features which are readily identified by visual inspection of the absorbent article. In prior art absorbent articles, leg elastic is commonly arranged between laterally extending side portions of a topsheet and a backsheet or between side portions of a standing gather material and the backsheet. Such arrangement results in a wrinkled, unesthetic and often relatively stiff and chafing non-elasticated side edge portion laterally outside of the elastic elements. When putting on an article having such non-elasticated side portions, it needs to be ascertained that the side-portions are folded out to avoid that the article is applied to the wearer with the elastic side portions folded in over the absorbent body, which may lead to poor fit and ultimately to leakage of body fluids out of the article.

An absorbent article as disclosed herein has soft, corrugated, bulky and well-defined leg cuffs which will automatically be correctly positioned when applying the article on a wearer without requiring any particular care or manipulation of the leg cuffs. The elasticated leg cuffs lie closely against the wearer's skin and form highly conformable and comfortable outer leakage barriers. By having the elastic elements distributed over the width of the leg cuffs, the risk of the leg cuffs exerting an inappropriately high pressure on the wearer's skin is considerably reduced or even eliminated.

In an absorbent article as disclosed herein, the side edge of the outer nonwoven cover layer is arranged inward of the leg cuff fold line in the nonwoven barrier cuff strip as seen in the transverse direction. By avoiding that the outer nonwoven cover layer extends all the way to the leg cuff fold line or even laterally outward of the fold line in the leg cuff fold, the side edge of the leg cuff of the absorbent article will be defined by the soft, resiliently deformable folded edge of the leg cuff fold.

In an absorbent article as disclosed herein, the outer nonwoven cover layer and/or the liquid impermeable backsheet may be attached to the leg cuff fold with an adhesive being uniformly distributed over the attachment area between the outer nonwoven cover layer and/or the liquid impermeable backsheet and the leg cuff fold or with uniformly distributed thermo-bonds or ultrasonic bonds. Accordingly, the adhesive is preferably uniformly distributed over the attachment area between the leg cuff portion of the nonwoven barrier cuff strip and the upper part of the side portion of the outer cover layer and/or of the backsheet. The adhesive may be applied by any suitable technique, such as slot-coating.

The corrugations which are formed in the leg cuffs by the elastic elements are visible both on the inner, wearer-facing side and on the outer garment-facing side of the leg cuffs.

### BRIEF DESCRIPTION OF THE DRAWINGS

The absorbent articles as disclosed herein will be further explained hereinafter with reference to the appended drawings wherein:

FIG. 1 shows a planar view of a stretched-out absorbent article in the form of a diaper, seen from the side which is intended to be facing a wearer during use of the diaper;

FIG. 2 shows a cross-section through the diaper in FIG. 1, taken along the line II-II in FIG. 1;

FIG. 3 shows a detail of a leg cuff;

FIG. 4 shows a detail of an alternative leg cuff;

FIG. 5 shows a detail of an alternative leg cuff,

FIG. 6 shows a detail of an alternative leg cuff; and

FIG. 7 shows a perspective view of the absorbent article in FIG. 1, ready for application on a wearer.

### DETAILED DESCRIPTION

Different aspects of the absorbent article as disclosed herein will be described more fully hereinafter with reference to the enclosed drawings. The embodiments disclosed herein should not be construed as being limiting to the claimed absorbent article.

It is to be understood that the drawings are schematic and that individual components, such as layers of material are not necessarily drawn to scale.

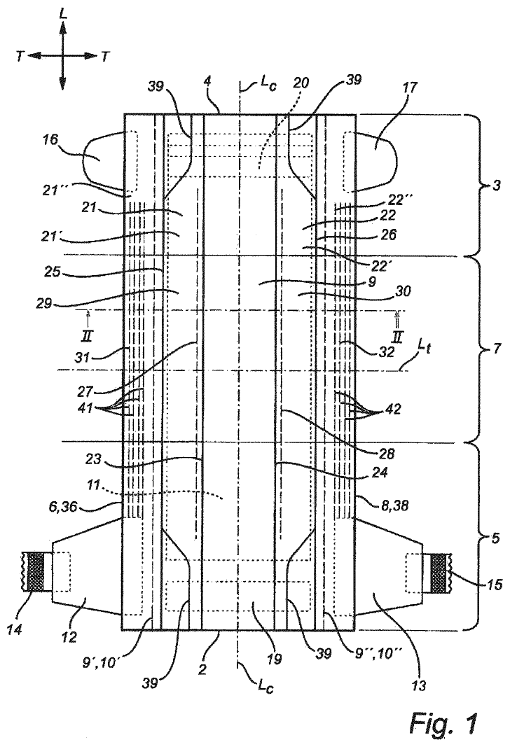

With reference to FIG. 1, there is shown an absorbent article 1 in the form of an open diaper. The absorbent article 1 is shown in FIG. 1 in an unfolded and flat condition with all elastic elements and components in an extended state, such that the absorbent article 1 appears generally two-dimensional. FIG. 1 shows the article from an inner, wearer-facing surface of the article. Any measurements of distances and dimensions of an actual absorbent article should be made with the absorbent article in the fully stretched-out, non-gathered condition as shown in FIG. 1.

The pant-type article 1 has a longitudinal direction L and a transverse direction T.

The absorbent article 1 has a first waist edge 2 and a second waist edge 4 and first and second side edges 6, 8. The first waist edge 2 of the absorbent article 1 which is shown in FIG. 1 is a rear waist edge and the second waist edge 4 of the absorbent article 1 is a front waist edge. The first and second waist edges 2, 4 extend in the transverse direction T of the absorbent article 1 and the side edges 6,8 extend in the longitudinal direction L of the absorbent article 1. The absorbent article 1 may be divided in the longitudinal direction L into a front portion 3, a rear portion 5 and a crotch portion 7 located between the front portion 3 and the rear portion 5.

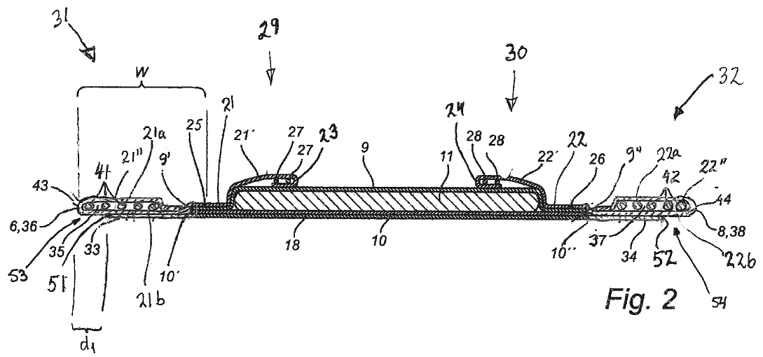

The absorbent article 1 comprises a liquid permeable topsheet 9 and a liquid impermeable backsheet 10. An absorbent core 11 is disposed between the topsheet 9 and the backsheet 10. The topsheet 9 has a topsheet first side edge 9′and a topsheet second side edge 9″ and the backsheet 10 has a backsheet first side edge 10′and a backsheet second side edge 10″, as indicated in FIGS. 1 and 2.

The absorbent article 1 further comprises a nonwoven outer cover layer 18 overlaying the backsheet 10 and forming an outer surface of the absorbent article 1.

A pair of nonwoven barrier cuff strips 21, 22 are arranged extending in the longitudinal direction L and are symmetrically arranged with one barrier cuff strip 21, 22 on each side of a longitudinal centre line Lc, perpendicular to a transverse centre line Lt. Each nonwoven barrier cuff strip 21, 22 comprises a standing gather portion 21′, 22′which is defined between a free standing gather edge 23, 24 extending in the longitudinal direction L and a standing gather hinge line 25, 26 extending in the longitudinal direction L. A pair of elasticated standing gathers 29, 30 comprising the standing gather portions 21′, 22′of the barrier cuff strips 21, 22 are arranged on the topsheet 9.

Each barrier cuff strip 21, 22 is directly attached to the topsheet 9 along the standing gather hinge line 25, 26 and has one or more pre-tensioned elastic elements 27, 28 arranged along the free standing gather edge 23, 24. The elastic element or elements may be elastic strings, threads or bands which are applied spaced apart along the free standing gather edge 23, 24. In an absorbent article as disclosed herein the number of elastic elements 27, 28 which are disposed along the standing gather edge, 23, 24 is typically 1, 2 or 3. The elastic elements 27, 28 are typically disposed parallel to each other with a spacing of from 2 to 5 millimetres, with an outermost elastic element arranged at the free standing gather edge 23, 24.

The absorbent article 1 further comprises a pair of leg cuffs 31, 32 extending in the longitudinal direction L and being symmetrically arranged along the first and second side edges 6, 8, each leg cuff 31, 32 being arranged outward of the standing gather portion 21′, 22′of the corresponding barrier cuff strip 21, 22.

As shown in FIG. 2, the leg cuffs 31, 32 each comprises a leg cuff portion 21″, 22″ of the corresponding barrier cuff strip 21, 22. The leg cuff portion 21″, 22″ of the nonwoven barrier cuff strip 21, 22 is defined in the transverse direction T between the standing gather hinge line 25, 26 and a leg cuff fold line 36, 38 along which the nonwoven barrier cuff strip 21, 22 is folded back on itself. A leg cuff fold 35, 37 is formed between a first part 21a, 22a and a second part 21b, 22b of the leg cuff portion 21″, 22″ of the barrier cuff strip 21, 22. The leg cuff fold 35, 37 has a leg edge coinciding with the side edge 6, 8 of the absorbent article 1 and with the fold line 36, 38. The width W of the leg cuff is defined in the transverse direction T between the leg edge of the leg cuff fold 35, 37 and the standing gather hinge line 25, 26.

In the embodiment shown in FIG. 2, the first part 21a, 22a of the leg cuff portion 21″, 22″ of each barrier cuff strip is an upper part of the leg cuff fold 35, 37 which is folded back along the leg cuff fold line 36, 38 and is attached to the second part 21b, 22b of the leg cuff portion 21″, 22″ of the barrier cuff strip 21, 22 on a wearer-facing side of the absorbent article 1.

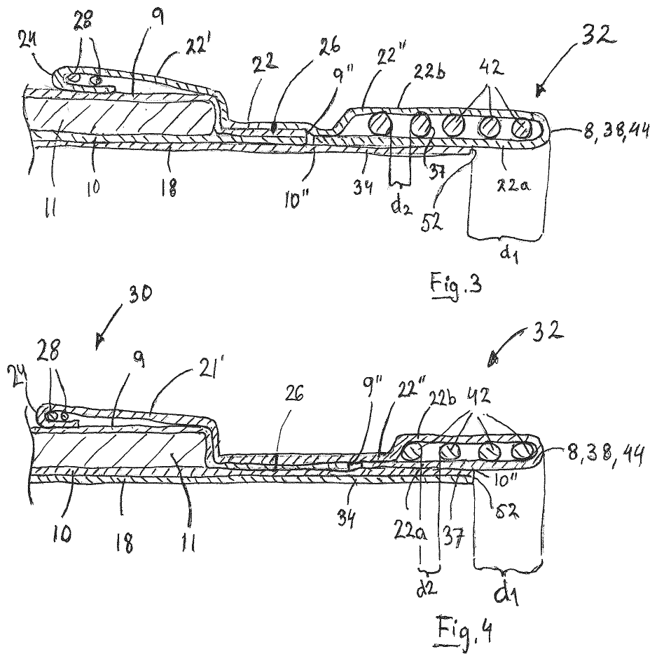

In an alternative folding variation, shown in FIGS. 3-5, the first part 21a, 22a of the leg cuff portion 21″, 22″ of each barrier cuff strip is a lower part of the leg cuff fold 35, 37 which is folded back along the leg cuff fold line 36, 38 and is attached to the second part 21b, 22b of the leg cuff portion 21″, 22″ of the barrier cuff strip 21, 22 on a garment-facing side of the absorbent article 1.

A plurality of spaced-apart pre-tensioned elastic elements 41, 42 are arranged in each leg cuff fold 35, 37. The elastic elements 41, 42 extend in the longitudinal direction L inside the leg cuff folds 35, 37 and are attached, e.g., by means of adhesive, between the first part 21a, 22a and the second part 21b, 22b of the leg cuff folds 35, 37.

A side portion 33, 34 of the outer cover layer 18 extends outward in the transverse direction T on each side of the absorbent core 11 and overlaps with the leg cuff portion 21″, 22″ of the nonwoven barrier cuff strip 21, 22. The side portions 33, 34 of the outer cover layer 18 are joined to the corresponding leg cuff fold 35, 37 with a side edge 51, 52 of the side portion 33, 34 of the outer cover layer 18 being arranged at a distance d1 from the leg cuff fold line 36, 38.

The parts of the leg cuff folds 35, 37 which are arranged laterally outward of the side edges 51, 52 of the side portions 33, 34 of the outer cover layer 18 constitute leg edge portions 53, 54 of the leg cuff folds 35, 37. The leg edge portions 53, 54 of the leg cuff folds 35, 37 each contain two of the elastic elements of the plurality of elastic elements 41, 42 in each leg cuff fold 35, 37. The leg edge portions 53, 54 are more flexible and conformable than the portions of the leg cuffs 31, 31 which are located laterally between the standing gather hinge lines 25, 26 and the side edge 51, 52 of the side portion 33, 34 of the outer cover layer 18. Thereby, the nonwoven material in the leg edge portions 53, 54 can be gathered in the longitudinal direction L by a lower contractive force than required for attaining the same degree of gathering in the portions of the leg cuff where the leg cuff fold is attached to the outer cover layer and/or to the liquid impermeable backsheet. Furthermore, while the elastic elements 41, 42 in the leg edge portions 53, 54 counteract complete collapse of the nonwoven material in the leg edge portions 53, 54, the leg edge portions 53, 54 of the leg cuff folds 35, 37 have a higher compressibility in the transverse direction T than the portion of the leg cuff where the leg cuff fold is attached to the outer cover layer and/or to the liquid impermeable backsheet. Thereby, the leg edge portion of the leg cuff provides lateral compressibility of the leg cuff which contributes to a cushioning effect against a wearer's legs.

FIG. 3 shows a portion of the absorbent article with the leg cuff 32 which is arranged along the second side edge 8. A plurality of elastic elements 42, represented by five individual elastic elements are arranged in the leg cuff fold 37 with a spacing d2. As set out above, the standing gather/leg cuff arrangement shown in FIG. 3 is the same as that in FIG. 2, with the only difference that the first part 22a of the leg cuff portion 22″ of the barrier cuff strip 22 constitutes a lower part of the leg cuff fold 37 instead of an upper part, as shown in FIG. 2.

The standing gather/leg cuff arrangement shown in FIG. 4 has the same leg cuff fold configuration as in FIG. 3 but differs from the standing gather/leg cuff arrangement in FIG. 3 in that the plurality of elastic elements 42 of the leg cuff fold 37 are only 4 instead of 5. The difference in number of elastic elements illustrates that the number of elastic elements may be varied within the ranges as disclosed herein, e.g., depending on the size of the article.

It is to be understood that although the elastic elements are shown to be distributed over the width of the leg cuff with an equal spacing between the elastic elements, the spacing d2 may be varied as disclosed herein e.g., to create a tighter fit in an inner part of the leg cuff than in the leg edge part of the leg cuff. Furthermore, the linear density and thereby generally also the diameter of the elastic elements may be different in different parts of the leg cuff, as disclosed herein.

A further difference between the standing gather/leg cuff arrangement shown in FIG. 4 and the standing gather/leg cuff arrangement shown in FIG. 3 is that the backsheet 10 in the FIG. 4 embodiment has the same width as the outer cover layer 18 with the second side edge 10″ of the backsheet 10 being located at the same distance d1 from the outer leg cuff side edge 44 as the side edge 52 of the outer cover layer 18. Such arrangement of the backsheet 10 and the outer cover layer 18 may be advantageous as the backsheet and outer cover layer may be separately produced as a backing laminate which is applied as a single unified component when producing an absorbent article as disclosed herein.

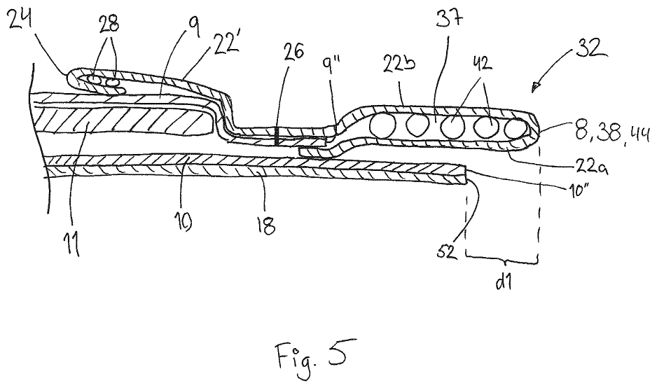

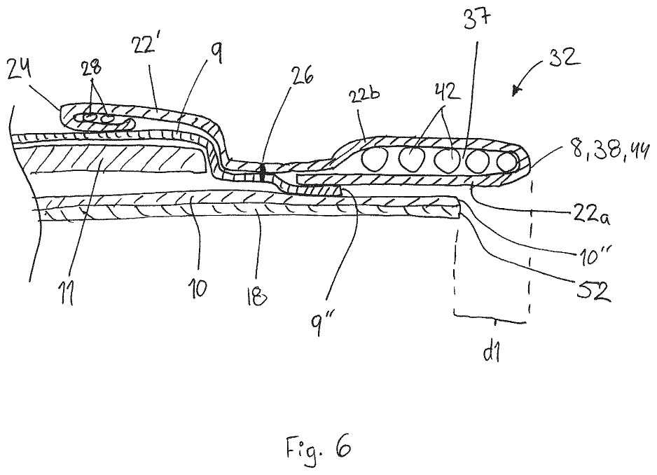

The standing gather/leg cuff arrangement shown in FIG. 5 has the same leg cuff fold configuration as in FIG. 4 but differs from the standing gather/leg cuff arrangement in FIG. 4 in that the plurality of elastic elements 42 of the leg cuff fold 37 are 5. Moreover, the leg cuff edge of the first part 22a of the leg cuff portion 22″ of the barrier cuff strip 22 is arranged underneath the liquid permeable topsheet 9. In this arrangement the liquid permeable topsheet 9, and the second side edge 9″ of the topsheet, overlaps a portion of the first part 22a of the leg cuff portion 22″ of the barrier cuff strip 22. The topsheet and the first part 22a of the leg cuff portion 22″ are attached to each other at the overlap by a longitudinal seal (not shown). The first part 22a of the leg cuff portion 22″ may further be attached to the liquid impermeable backsheet 10.

The standing gather/leg cuff arrangement shown in FIG. 6 has the same leg cuff fold configuration as in FIG. 5 but differs in that the leg cuff edge of the first part 22a of the leg cuff portion 22″ of the barrier cuff strip 22 is arranged on top of the liquid permeable topsheet 9. In other words, a portion of the first part 22a of the leg cuff portion 22″ is arranged in between the topsheet 9 and the second part 22b of the leg cuff portion 22″.

The topsheet 9 and the first part 22a of the leg cuff portion 22″ are attached to each other at the overlap by a longitudinal seal (not shown). The first part 22a of the leg cuff portion 22″ may further be attached to the second part 22b of the leg cuff portion 22″.

In FIG. 5 and FIG. 6 the liquid impermeable backsheet 9 and the nonwoven outer cover has the same width but may also be of different width in combination with the topsheet 9 and leg cuff portion 22a overlapping.

For all embodiments, the width of the leg cuffs 31, 32 may be in the range of from 20 millimetres to 60 millimetres and the elastic elements of the plurality of elastic elements 41, 42 may be distributed over an area of each leg cuff having a width which is at least 40% of the width of the leg cuff, preferably at least 50% of the width of the leg cuff. The width of the elasticated portion of the leg cuffs 31, 32 may be in the range of from 20 millimetres to 40 millimetres.

The elastic elements of the plurality of elastic elements 41, 42 in each leg cuff 31, 32 may suitably be attached between the first and second parts 21a 22a; 21b, 22b of the leg cuff folds 35, 37 by means of adhesive. Adhesive may be applied to the elastic elements, and/or to one or both of the first and second parts 21a 22a; 21b, 22b of the leg cuff fold 35, 37. Alternatively, the elastic elements may be anchored in the leg cuff fold 35, 37 by means of heat welding or ultrasonic welding, e.g., as disclosed in WO2021/037352 A1.

The elastic elements may be elastic threads, bands, strings, etc. All elastic elements may be of the same type and having the same elastic properties. Alternatively, one or more elastic elements being arranged in an inner part of the leg cuff fold 35, 37 may have a greater tensile resistance than one or more elastic elements being arranged in an outer part of the leg cuff fold 35, 37. By way of example, the elastic elements in the leg edge portions 53, 54 of the leg cuff folds 35, 37 may have a lower tensile resistance than the other elastic elements in the leg cuff folds 35, 37.

The one or more elastic elements in the inner part of the leg cuff fold 35, 37 may have a linear density (dtex) which is higher than the linear density of the one or more elastic elements being arranged in the outer portion of the leg cuff fold 35, 37. By way of example, the elastic elements in the leg edge portions 53, 54 of the leg cuff folds 35, 37 may have a linear density that is lower than the linear density of the other elastic elements in the leg cuff folds 35, 37.

With reference to FIG. 1, a first side flap 12 is arranged on the rear portion 5 of the absorbent article 1, at the rear waist edge 2, and extends from the first side edge 6 in the transverse direction T of the absorbent article 1 and a second side flap 13 is arranged on the rear portion 5, at the rear waist edge 2, and extends from the second side edge 8 in the transverse direction T of the absorbent article 1. The first and second side flaps 12, 13 are attachment flaps and are provided with fasteners 14, 15 for securing the absorbent article 1 in a pant-like configuration about the lower trunk of a wearer's body. Such attachment flaps are optional features of an absorbent article as disclosed herein e.g., in absorbent articles being pant diapers or belted diapers.

The first and second side flaps 12, 13 are tensioned when putting on the absorbent article and need to be firmly anchored along the side edges 6,8 to ascertain that they do not come off the article when exposed to laterally applied pulling forces. The first and second side flaps 12, 13 may be made from any suitable material known in the art, such as fibrous nonwoven material. The first and second side flaps 12, 13 may be elastic or may be elastically stretchable, at least in the lateral direction of the absorbent article 1. The first and second side flaps 12, 13 may be breathable and are preferably soft and pliable for providing good body conformance and comfort.

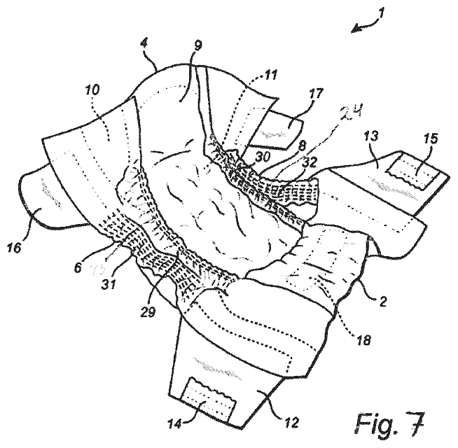

The first and second side flaps 12, 13 which are shown in FIGS. 1 and 7 are provided with fastener elements 14, 15 which may be of any suitable kind such as adhesive or mechanical fasteners. A common type of mechanical fastener is hook-and-loop fasteners.

In the absorbent article 1 in FIGS. 1 and 7, the fastener elements 14, 15 are shown as hook-type fasteners, i.e., fasteners having multiple protruding elements which may engage with a corresponding loop-type fastener such as a loop-type nonwoven material. A loop-type fastener may be provided by a portion of the nonwoven outer cover layer 18 which overlies the backsheet 10 on the outer, garment-facing, surface of the absorbent article 1. Alternatively, a dedicated fastener receiving area 20 formed by a fastener receiving material maybe be arranged on the outer, garment-facing surface of the front portion 3 of the absorbent article 1, as indicated in FIG. 1.

The fastener elements 14, 15 may alternatively be adhesive fastener tapes which are arranged to be attached on a receiving area arranged on the front portion 3 of the absorbent article 1. A further option is that the first and second side flaps 12, 13 are arranged to be attached to third and fourth side flaps arranged at the opposite waist edge. Furthermore, the first and second side flaps 12, 13 may have a lateral extension which is large enough such that the first and second side flaps 12, 13 can be fastened to each other in the manner of a belt. It is further to be understood that in a hook-and-loop type fastener arrangement, the hook-type fastener elements may be arranged on a receiving surface while the loop type fastener elements are arranged on a first and/or a second side flap.

The third and fourth side flaps 16,17 as shown in the figures are optional to the absorbent article as disclosed herein. The third and fourth side flaps 16,17, may be made from generally the same materials as the first and second side flaps 12, 13. However, elastic materials are less suitable for use in the third and fourth side flaps 16, 17. In the absorbent article 1 in FIGS. 1 and 7, the third and fourth side flaps 16,17 are positioning flaps serving as grip members when applying the front portion 3 of the absorbent article 1 over a wearer's belly.

As is best seen in FIG. 1, the plurality of elastic elements 41, 42 in the leg cuffs 31, 32 preferably extend only along the parts of the side edges 6, 8 which are located between the first and third side flaps 12, 16 for the first side edge 6 and between the second and fourth side flaps 13, 15 for the second side edge 8. This means that the major parts of the leg cuffs 31, 32 are located in the crotch portion 7 of the absorbent article 1. Likewise, the elastic elements 27, 28 in the standing gathers 29, 30 are preferably arranged as shown in FIG. 1 such that the elastic elements 27, 28 do not extend all the way to the end edges 2, 4 of the absorbent article 1. The non-elasticated parts of the standing gathers 29,30 are secured to the topsheet along lines of securement 39. Thereby, under the influence of the elastic forces in the elastic elements 27, 28, the portion of the absorbent article 1 which is located between the standing gathers 29, 30 assumes a pouch-shaped in-use configuration in the crotch portion 7, as shown in FIG. 7.

The absorbent article 1 in FIGS. 1 and 7 further includes an optional waist elastic member 18. The waist elastic member 19 is located at the rear waist edge 2 and extends in the lateral direction along only a part of the rear waist edge 2. Any type of waist elastic may be used, as known in the art. Furthermore, the absorbent article 1 may include waist elastic also at the front portion 3 of the absorbent article 1.

As seen in FIG. 7, when the absorbent article 1 has been taken out of a package and has been folded out ready for application to a wearer, the standing gathers 29, 30 are raised up from the topsheet 9 as a result of the contractive forces exerted on the free standing gather edges 23, 24 by the pre-tensioned elastic elements 27, 28 which are arranged on the standing gathers 29, 30. The elastic elements of the plurality of elastic elements 41, 42 in the leg cuffs 31, 32 gather the nonwoven material in the leg cuff folds 35, 37, thereby creating fluffy and breathable leg cuffs 31, 32, which have a broad skin-contacting inner surface over which the contractive forces from the plurality of elastic elements 41, 42 in the leg cuffs 31, 32 are distributed. The leg cuffs 31, 32 are resiliently compressible in a thickness direction of the leg cuffs 31, 32. Due to the provision of the leg edge portions 53, 54, the leg cuffs 31, 32 are also resiliently compressible in the lateral or transverse direction. Thus, the leg cuffs fit closely around the legs of a wearer in a comfortable manner, with virtually no risk of chafing or other discomforts being caused by the leg cuffs.

As further seen in FIG. 7, the broad, generally band-shaped, elasticated leg cuffs 31, 32 are bulky and shape stable and automatically assumes a correct position when the absorbent article is applied to a wearer.

The absorbent core 11 is disposed between the topsheet 9 and the backsheet 10 and is arranged to absorb liquid, such as urine or other body fluids, which has passed through the topsheet 9. The absorbent core 11 may be a single-layer structure or may be a layered structure. The absorbent core 11 may comprise any conventional material suitable for absorbing discharged bodily wastes, such as cellulosic fluff pulp, tissue layers, highly absorbent polymers (super absorbents), absorbent foam materials including hydrogel-foam material, nonwoven materials, or the like. The absorbent core may comprise non-absorbent components such as stiffening elements, shaping elements, binders, etc. Various types of liquid-receiving and liquid distribution materials can also be included in the core.

The absorbent core 11 may be a three-dimensionally shaped core with different amounts of absorbent material in different parts of the core. The core may be provided with features such as embossings, fluid acquisition cavities and fluid distribution channels.

Properties of the absorbent core such as density, amounts of absorbent material and absorbent capacity may also be varied in different portions of the absorbent core. The absorbent core 11 may comprise a core cover, sandwiching absorbent material between an upper core cover layer and a lower core cover layer. Such core cover may be a tissue material or a nonwoven material and is provided in addition to the topsheet 9 and the backsheet 10.

The liquid permeable topsheet 9 may comprise or consist of a nonwoven material. Other suitable topsheet materials include tow fibres, porous foams, apertured plastic films and laminates and combinations of such materials. The materials which are best suited as topsheet materials are soft and non-irritating to the skin, are readily penetrated by body fluids, and display low rewet of absorbed body fluid.

The liquid impermeable backsheet 10 may be any conventional liquid impermeable material such as a thin plastic film, e. g. a polyethylene or polypropylene film. The backsheet material may be breathable to allow vapour to escape from the absorbent body, while still preventing liquids from passing through the backsheet material.

1. An absorbent article having a first waist edge and a second waist edge extending in a transverse direction of the absorbent article and a first side edge and a second side edge extending in a longitudinal direction of the absorbent article, the absorbent article comprising:
a liquid permeable topsheet;
a liquid impermeable backsheet;
an absorbent core disposed between the topsheet and the backsheet;
a nonwoven outer cover layer overlaying the backsheet and forming an outer surface of the absorbent article;
a pair of nonwoven barrier cuff strips extending in the longitudinal direction and being symmetrically arranged with one barrier cuff strip on each side of a longitudinal centre line, each nonwoven barrier cuff strip comprising a standing gather portion, and a leg cuff portion the standing gather portion being defined between a free standing gather edge extending in the longitudinal direction and a standing gather hinge line extending in the longitudinal direction, the barrier cuff strip being attached to the topsheet along the standing gather hinge line, and having one or more pre-tensioned elastic elements arranged along the free standing gather edge;
a pair of standing gathers comprising the standing gather portions of the barrier cuff strips;
a pair of leg cuffs extending in the longitudinal direction and being symmetrically arranged along the first and second side edges, each leg cuff being arranged outward of the standing gather portion of the corresponding barrier cuff strip in the transverse direction; and
wherein each leg cuff comprises:
the leg cuff portion of the corresponding nonwoven barrier cuff strip, the leg cuff portion of the nonwoven barrier cuff strip extending in the transverse direction between the standing gather hinge line and a leg cuff fold line along which the nonwoven barrier cuff strip is folded back on itself and forms a leg cuff fold, the leg cuff fold line extending in the longitudinal direction and dividing the leg cuff portion of the barrier cuff strip in a first part and a second part which first and second parts together form the leg cuff fold
a plurality of spaced-apart pre-tensioned elastic elements extending in the longitudinal direction inside the leg cuff fold and being attached between the first part and the second part of the leg cuff fold; and
a side portion of the outer cover layer overlapping with the leg cuff portion of the nonwoven barrier cuff strip and being joined to the leg cuff fold with a side edge of the side portion of the outer cover layer being arranged at a distance from the leg cuff fold line.

2. The absorbent article according to claim 1, wherein the first part of the leg cuff portion of each barrier cuff strip is folded back along the leg cuff fold line and is attached to the second part of the leg cuff portion of the barrier cuff strip on a wearer-facing side of the absorbent article.

3. The absorbent article according to claim 1, wherein the first of the leg cuff portion of each barrier cuff strip is folded back along the leg cuff fold line and is attached to the second part of the leg cuff portion of the barrier cuff strip on a garment-facing side of the absorbent article.

4. The absorbent article according to claim 3, wherein an inner portion of the first part of the leg cuff portion of each barrier cuff strip overlaps the liquid permeable topsheet such that the leg cuff edge of the leg cuff portion of each barrier cuff strip is arranged inwards in relation to the side edge of the topsheet in transverse direction.

5. The absorbent article according to claim 1, wherein a leg edge portion of each leg cuff fold being located between the side edge of the side portion of the outer cover layer and the leg cuff fold line contains two or more of the elastic elements of the plurality of elastic elements in the leg cuff fold.

6. The absorbent article according to claim 1, wherein the width of each leg cuff as measured in the transverse direction from the standing gather hinge line to the leg cuff fold line is in the range of from 20 millimetres to 60 millimetres.

7. The absorbent article according to claim 6, wherein the elastic elements of the plurality of elastic elements in each leg cuff are distributed over an area of the leg cuff having a width in the transverse direction which is at least 40% of the width of the leg cuff.

8. The absorbent article according to claim 1, wherein the plurality of elastic elements in each leg cuff constituted by at least 3 elastic elements.

9. An The absorbent article according to claim 8, wherein the elastic elements are spaced apart in the transverse direction with a spacing being in the range from 1 mm to 8 mm.

10. The absorbent article according to claim 1, wherein the liquid impermeable backsheet and the nonwoven outer cover layer have the same width in the transverse direction.

11. The absorbent article according to claim 10, wherein there is no overlap between the leg cuff folds and the topsheet and the leg cuff folds are directly attached to the liquid impermeable backsheet.

12. The absorbent article according to claim 1, wherein the nonwoven. outer cover layer has a greater extension in the transverse direction than the liquid impermeable backsheet.

13. The absorbent article according to claim 12, wherein there is no overlap between the leg cuff folds and the topsheet and the leg cuff folds are directly attached to the side portions of the outer cover layer.

14. The absorbent article according to claim 1, wherein all or substantially all elastic elements of the plurality of elastic elements in each leg cuff fold are arranged in a portion of the leg cuff fold which is free from overlap with the liquid impermeable backsheet.

15. The absorbent article according to claim 4, wherein the leg edge portion is arranged at outside of the liquid impermeable backsheet in the transverse direction.

16. The absorbent article according to claim 1, wherein the nonwoven barrier cuff strip is a single layer nonwoven material.

17. The absorbent article according to claim 1, wherein the elastic elements of the plurality of elastic elements are attached between the first and second parts of the leg cuff fold by means of adhesive.

18. The absorbent article according to claim 1 wherein one or more elastic elements being arranged in an inner part of the leg cuff fold has/have a greater tensile resistance than one or more elastic elements being arranged in an outer part of the leg cuff fold.

19. An The absorbent article according to claim 18, wherein the one or more elastic elements in the inner part of the leg cuff fold has a linear density which is higher than a linear density of the one or more elastic elements being arranged in the outer portion of the leg cuff fold.

20. The absorbent article according to claim 1, wherein the absorbent article further comprises a pair of side flaps arranged at one of the first or second waist edges, each side flap extending in the transverse direction from a proximal attached end to a free distal end and comprising a fastener being arranged at the distal end of the side flap.
